(19) United States
(12) Patent Application Publication
(10) Pub. No.: US 2022/0141267 A1
(43) Pub. Date: May 5, 2022

(54) Tele-Orthodontic System

(71) Applicants: Ricky Eugene Harrell, Atlanta, GA (US); Gasper Lazarra, Ponte Vedra, FL (US); Chris Wynne, Ponte Vedra Beach, FL (US)

(72) Inventors: Ricky Eugene Harrell, Atlanta, GA (US); Gasper Lazarra, Ponte Vedra, FL (US); Chris Wynne, Ponte Vedra Beach, FL (US)

(21) Appl. No.: 17/519,010

(22) Filed: Nov. 4, 2021

Related U.S. Application Data
(60) Provisional application No. 63/110,141, filed on Nov. 5, 2020.

(51) Int. Cl. H04L 65/403; H04L 65/1096; H04N 5/225; G16H 40/67; G16H 80/00

(52) U.S. Cl. CPC H04L 65/403; H04L 65/1096; H04N 2005/2255; G16H 40/67; G16H 80/00; H04N 5/225

(57) ABSTRACT

A tele-orthodontic system and its methodology wherein a remotely located orthodontist/faculty member, via two-way video and audio communication, can communicate with and evaluate a patient in a clinic and can further advise an assistant/resident present with the patient in the performance of necessary procedures.

### BACKGROUND OF THE INVENTION

This invention relates generally to systems and methods for the remote provision of medical services, such as for example examination, diagnosis, treatment instructions, etc., wherein the treating professional is at a location remote and separated from the location of the patient, and wherein various forms of telecommunications, both audio and visual, are utilized. Such systems and methods are often referred to as tele-medicine. More particularly, the invention relates to such systems and methods wherein a second professional is physically present with the patient. Even more particularly, the invention relates to such systems in the field of orthodontics.

Tele-medicine as a broad concept is known, however the concept has not been optimally applied in the field of orthodontics, and as such there is a need for a novel and non-obvious system and method particularly focusing on the field of orthodontics. As used herein, the term “orthodontist” and similar variations shall be taken to include dentists, orthodontists and similar professionals, and the term “orthodontic” and similar variations shall be taken to include dental, orthodontic and similar practices.

### BRIEF DESCRIPTION OF THE DRAWINGS

FIG. 1A, in combination with FIGS. 1B and 1C, illustrates a portion of a representative embodiment of the tele-orthodontic system in flowchart form, the embodiment in total showing a plurality of virtual clinics, a telecommunication and software processing network and a plurality of remote orthodontist/faculty members.

FIG. 1B, in combination with FIGS. 1A and 1C, illustrates a portion of a representative embodiment of the tele-orthodontic system in flowchart form, the embodiment in total showing a plurality of virtual clinics, a telecommunication and software processing network and a plurality of remote orthodontist/faculty members.

FIG. 1C, in combination with FIGS. 1A and 1B, illustrates a portion of a representative embodiment of the tele-orthodontic system in flowchart form, the embodiment in total showing a plurality of virtual clinics, a telecommunication and software processing network and a plurality of remote orthodontist/faculty members.

### SUMMARY OF THE INVENTION

The invention in various embodiments comprises a system for the practice of tele-orthodontics in a private practice and educational residency setting wherein the methodology includes the physical presence of a patient in one of one or more clinics along with an assistant (in a private practice) or a resident (in an educational residency program). The assistant/resident sees active treatment patients in the clinic. The orthodontist (in a private practice) or an orthodontic faculty member (in an educational residency program) are not physically located in the clinic but are using a computer, phone, or tablet in combination with audio and visual telecommunication equipment to communicate with the assistant and resident via orthodontic clinic software from a remote location, which creates in effect a virtual orthodontic clinic.

Alternatively summarizing, the invention is a tele-orthodontic system comprising: a plurality of orthodontic clinics, an orthodontic assistant or resident physically present within each of the plurality of orthodontic clinics, and a patient physically present within each of the plurality of orthodontic clinics; each of the plurality of orthodontic clinics containing a live video communication device, a live video and audio communication device and a live video, written and audio communication device; a communication and processing network utilizing real-time video and audio cloud online software to create and maintain a plurality of virtual waiting rooms, each of the virtual waiting rooms being dedicated to a particular orthodontist/faculty member located geographically remotely from any of the plurality of orthodontic clinics; each of the geographically remotely located orthodontists/faculty members possessing a remote live video and audio communication device and a remote live video, written and audio communication device; wherein the live video communication device, a live video and audio communication device and a live video, written and audio communication device of the plurality of clinics is in two-way communication through the communication and processing network with the remote live video and audio communication device and a remote live video, written and audio communication device of the geographically remotely located orthodontists/faculty members; such that the geographically remotely located orthodontists/faculty members may analyze the communications from the plurality of orthodontic clinics and provide instruction to the orthodontic assistants or residents present in the plurality of clinics for procedures to be performed on the patients. Furthermore, such invention wherein the live video and audio communication device and the live video, written and audio communication devices within each of the plurality of orthodontic clinics is encompassed in a single device; wherein the remote live video and audio communication device and the remote live video, written and audio communication devices possessed by one of the geographically remotely located orthodontists/faculty members is encompassed in a single device; wherein live video communication device within each of the plurality of clinics is an intraoral or digital camera and/or wherein each of the virtual waiting rooms is dedicated to a different one of the plurality of clinics.

### DETAILED DESCRIPTION OF THE INVENTION

With reference to the figures, embodiments of the invention are now described in detail. The invention in various embodiments comprises a system for the practice of tele-orthodontics in a private practice and educational residency setting wherein the methodology includes the physical presence of a patient in one of one or more clinics along with an assistant (in a private practice) or a resident (in an educational residency program). The assistant/resident sees active treatment patients in the clinic. The orthodontist (in a private practice) or an orthodontic faculty member (in an educational residency program) are not physically located in the clinic but are using a computer, phone, or tablet in combination with audio and visual telecommunication equipment to communicate with the assistant and resident via orthodontic clinic software from a remote location, which creates in effect a virtual orthodontic clinic. The system and method thus provides the remote orthodontist/faculty member with a virtual waiting room, HIPAA compliant audio and video communication, and document transmission. The use of a virtual orthodontic clinic with a virtual waiting room allows the orthodontist/faculty member to control patient appointments and workflow in the clinic, and further allows a single orthodontist/faculty member to be in successive communication with multiple virtual clinics at different physical locations.

The assistant is equipped with an internet connected computer, tablet, or phone, a computer connected intraoral camera or digital camera, and headphones with a microphone. The assistant/resident use the intraoral or digital camera and headphones connected to a computer to communicate with the orthodontist/faculty member using virtual orthodontic clinic software. The assistant/resident clicks the virtual waiting room link on the chairside computer when the patient is prepped and ready to see and be seen by the orthodontist/faculty member. The assistant enters the patient's name in the virtual waiting room software, which places the patient in a queue in the virtual waiting room. When the orthodontist/faculty member is ready to see their next patient, the clinic software initiates the telecommunication with the next patient name in the virtual waiting room.

The virtual orthodontic clinic software allows the orthodontist/faculty member to see live video, still pictures, or recorded videos of the patient's mouth. The orthodontist/faculty member use the videos, pictures, recordings to evaluate the patient's treatment progress and to instruct the assistant/resident via the virtual orthodontic clinic software (verbally, in writing, or by recording) on the procedures that need to be performed on that patient at that appointment.

This system utilizes hardware, software and Internet communications to serve patients physically located in the clinic alongside an assistant or resident, with an orthodontist/faculty member supervising from a distance. The procedures carried out with the tele-orthodontics will be reversible procedures only. All irreversible procedures must be carried out by the orthodontist in person.

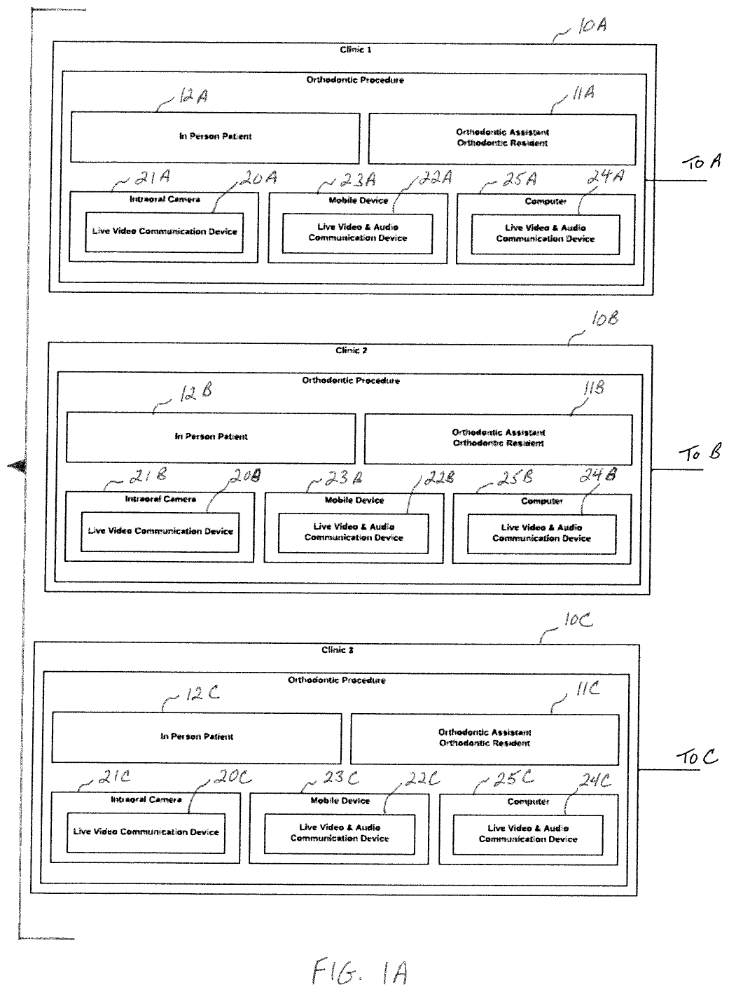

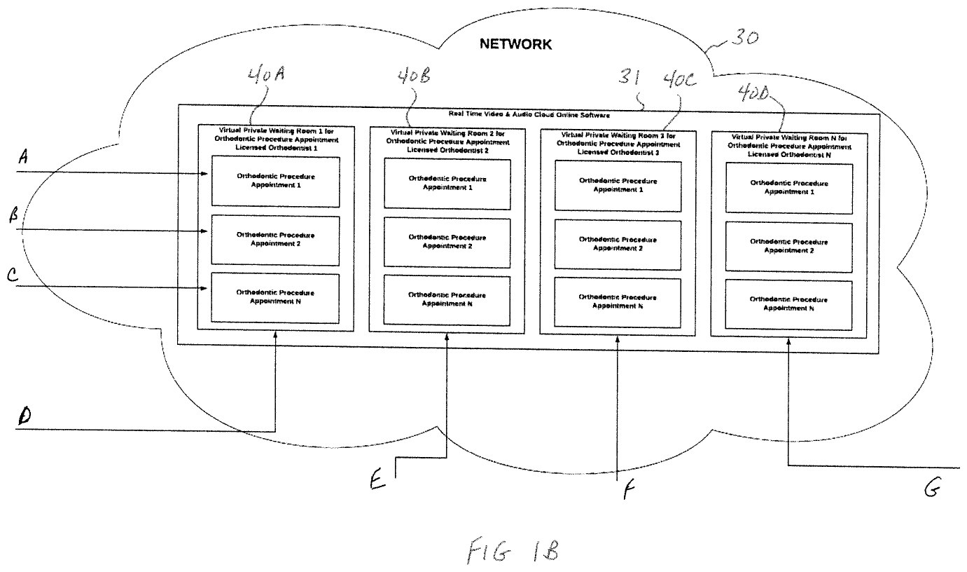

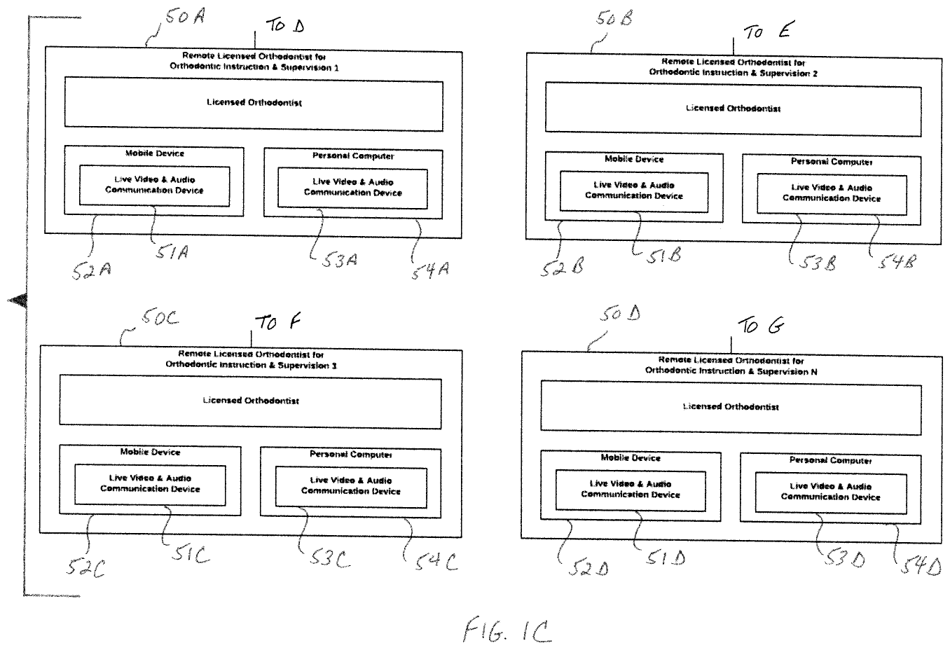

FIGS. 1A, 1B and 1C are presented on separate pages but are to be reviewed in combination, such that the three FIGS. 1A, 1B and 1C illustrate a full embodiment of the system and its methodology.

FIG. 1A illustrates a portion of an embodiment of the system and its methodology comprising a plurality of clinics 10A/10B/10C. Each representative clinic 10A/10B/10C handling an orthodontic procedure possesses a live video communication device 20A/20B/20C, such as an intraoral camera or digital camera 21A/21B/21C, a live video and audio communication device 22A/22B/22C, such as a mobile device 23A/23/B/23C, and a live video, written and audio communication device 24A/24B/24C, such as a computer 25A/25B/25C, all to provide communication between the clinic 10A/10B/10C to a remote location. An orthodontic assistant or resident 11A/11B/11C is physically present in the clinic 10A/10B/10C with a patient 12A/12B/12C. Live video and audio communication device 22A/22B/22C and live video, written and audio communication device 24A/24B/24C may be encompassed in a single instrument, such as a smart cell phone. Each clinic 10A/10B/10C communicates with a communication and processing network 30, as shown in FIG. 1B.

FIG. 1B illustrates a portion of an embodiment of the system and its methodology comprising a communication and processing network 30 which utilizes real-time video and audio cloud online software 31 to create and maintain a plurality of virtual waiting rooms 40A/40B/40C/40D each dedicated to an orthodontist/faculty member 50A/50B/50C/50D, as shown in FIG. 1C. Patients in a particular waiting room 40A/40B/40C/40D may come from a single clinic 10A/10B/10C or from multiple clinics 10A/10B/10C. The communication and processing network 30 receives and transmits communication between the clinics 10A/10B/10C and the remotely located orthodontists/faculty members 50A/50B/50C/50D.

If the patient 12A/12B/12C is a dedicated patient to a particular remotely located orthodontist/faculty member 50A/50B/50C/50D, the patient 12A/12B/12C is placed in the virtual waiting room 40A/40B/40C/40D of the dedicated orthodontist/faculty member 50A/50B/50C/50D. If the patient 12A/12B/12C is not a dedicated patient to a particular remotely located orthodontist/faculty member 50A/50B/50C/50D, the communication and processing network 30 may place the patient 12A/12B/12C into the virtual waiting room 40A/40B/40C/40D of any dedicated orthodontist/faculty member 50A/50B/50C/50D.

FIG. 1C illustrates a portion of an embodiment of the system and its methodology comprising a plurality of geographically remotely located orthodontists/faculty members 50A/50B/50C/50D. Each orthodontist/faculty member 50A/50B/50C/50D handling an orthodontic procedure possesses a remote live video and audio communication device 51A/51B/51C/51D, such as a mobile device 52A/52B/52C/52D, and a remote live video, written and audio communication device 53A/53B/53C/53D, such as a computer 54A/54B/54C/54D. Remote live video and audio communication device 51A/51B/51C/51D and remote live video, written and audio communication device 53A/53B/53C/53D may be encompassed in a single device, such as mobile cell phone or a computer laptop. Each remotely located orthodontist/faculty member 50A/50B/50C/50D communicates with a communication and processing network 30, as shown in FIG. 1B.

In this manner each clinic 10A/10B/10C is in two-way communication with each remotely located orthodontist/faculty member 50A/50B/50C/50D, such that video information for a patient 12A/12B/12C, obtained by the orthodontic assistant or resident 11A/11B/11C is delivered to and analyzed by the remotely located orthodontist/faculty member 50A/50B/50C/50D. The remotely located orthodontist/faculty member 50A/50B/50C/50D then instructs the orthodontic assistant or resident 11A/11B/11C to perform a procedure if required and/or provides instructions or explanations to the patient 12A/12B/12C.

As a representative example of the system and method, a patient 12A having braces mounted onto their teeth may schedule and attend an appointment at a clinic 10A, where the assistant/resident 11A preps the patient 12A as needed for telecommunication with the orthodontist/faculty member 50A and communicates the necessary information into the network 30. When the orthodontist/faculty member 50A is ready to examine the patient 11A the two-way video and audio communication is initiated between the clinic 10A and the remote orthodontist/faculty member 50A. The assistant/resident 11A uses the intraoral camera 21A and/or digital camera to provide visuals of the patient's oral cavity to the orthodontist/faculty member 50A for evaluation of the condition of the braces, progression in treatment, etc. The orthodontist/faculty member 50A can then instruct the assistant/resident 11A to, for example tighten or re-adjust the braces. The patient 12A can ask any questions of the orthodontist/faculty member 50A, and the orthodontist/faculty member 50A can instruct the patient 12A as needed.

It is understood that equivalents and substitutions for certain elements set forth above may be obvious to those of skill in the art, and therefore the true scope and definition of the invention is to be as set forth in the following claims.

We claim:

1. A tele-orthodontic system comprising:
a plurality of orthodontic clinics, an orthodontic assistant or resident physically present within each of the plurality of orthodontic clinics, and a patient physically present within each of the plurality of orthodontic clinics; each of the plurality of orthodontic clinics containing a live video communication device, a live video and audio communication device and a live video, written and audio communication device;
a communication and processing network utilizing real-time video and audio cloud online software to create and maintain a plurality of virtual waiting rooms, each of the virtual waiting rooms being dedicated to a particular orthodontist/faculty member located geographically remotely from any of the plurality of orthodontic clinics;
each of the geographically remotely located orthodontists/faculty members possessing a remote live video and audio communication device and a remote live video, written and audio communication device;
wherein the live video communication device, a live video and audio communication device and a live video, written and audio communication device of the plurality of clinics is in two-way communication through the communication and processing network with the remote live video and audio communication device and a remote live video, written and audio communication device of the geographically remotely located orthodontists/faculty members;
such that the geographically remotely located orthodontists/faculty members may analyze the communications from the plurality of orthodontic clinics and provide instruction to the orthodontic assistants or residents present in the plurality of clinics for procedures to be performed on the patients.

2. The tele-orthodontic system of claim 1 wherein the live video and audio communication device and the live video, written and audio communication devices within each of the plurality of orthodontic clinics is encompassed in a single device.

3. The tele-orthodontic system of claim 1 wherein the remote live video and audio communication device and the remote live video, written and audio communication devices possessed by one of the geographically remotely located orthodontists/faculty members is encompassed in a single device.

4. The tele-orthodontic system of claim 1 wherein the live video and audio communication device and the live video, written and audio communication devices within each of the plurality of orthodontic clinics is encompassed in a single device, and
wherein the remote live video and audio communication device and the remote live video, written and audio communication devices possessed by one of the geographically remotely located orthodontists/faculty members is encompassed in a single device.

5. The tele-orthodontic system of claim 1 wherein the live video communication device within each of the plurality of clinics is an intraoral or digital camera.

6. The tele-orthodontic system of claim 1 wherein each of the virtual waiting rooms is dedicated to a different one of the plurality of clinics.

7. The tele-orthodontic system of claim 6 wherein the live video and audio communication device and the live video, written and audio communication devices within each of the plurality of orthodontic clinics is encompassed in a single device.

8. The tele-orthodontic system of claim 6 wherein the remote live video and audio communication device and the remote live video, written and audio communication devices possessed by one of the geographically remotely located orthodontists/faculty members is encompassed in a single device.

9. The tele-orthodontic system of claim 6 wherein the live video and audio communication device and the live video, written and audio communication devices within each of the plurality of orthodontic clinics is encompassed in a single device, and
wherein the remote live video and audio communication device and the remote live video, written and audio communication devices possessed by one of the geographically remotely located orthodontists/faculty members is encompassed in a single device.

10. The tele-orthodontic system of claim 6 wherein the live video communication device within each of the plurality of clinics is an intraoral or digital camera.
